(19) United States
(12) Patent Application Publication
(10) Pub. No.: US 2019/0022376 A1
(43) Pub. Date: Jan. 24, 2019

(54) ACTIVE RETINA IMPLANT

(71) Applicant: NMI Naturwissenschaftliches und Medizinisches Institut an der Universität Tübingen, Reutlingen (DE)

(72) Inventors: Günther Zeck, Reutlingen (DE); Thoralf Herrmann, Tübingen (DE); Florian Jetter, Reutlingen (DE); Alfred Stett, Reutlingen (DE)

(73) Assignee: NMI Naturwissenschaftliches und Medizinisches Institut an der Universität Tübingen

(21) Appl. No.: 16/137,452

(22) Filed: Sep. 20, 2018

Related U.S. Application Data
(63) Continuation of application No. PCT/EP2017/055802, filed on Mar. 13, 2017.

(30) Foreign Application Priority Data
Mar. 21, 2016 (DE) 102016105174.8

(51) Int. Cl. A61N 1/05; A61N 1/36

(52) U.S. Cl. CPC A61N 1/0543; A61N 5/0622; A61N 1/36046

(57) ABSTRACT

The present disclosure relates to an active retinal implant for implantation in an eye, comprising an array of stimulation electrodes that deliver stimulation signals to retinal cells. At least one signal generator is provided which generates at least one continuous sinusoidal stimulation signal that comprises at least one adjustable signal parameter, and the at least one signal generator is electrically coupled to at least one stimulation electrode.

### CROSS REFERENCE TO RELATED APPLICATIONS

This is a U.S. bypass continuation application of co-pending international patent application PCT/EP2017/055802, filed Mar. 13, 2017 and designating the United States, which was published in German as WO 2017/162458 A1, and claims priority to German patent application DE 10 2016 105 174.8, filed Mar. 21, 2016, which is incorporated herein by reference.

### BACKGROUND

This invention relates to an active retinal implant for implantation in an eye, with an array of stimulation electrodes that deliver stimulation signals to retinal cells.

An exemplary retinal implant is described in WO 2005/000395 A1.

The retinal implant serves to counteract vision loss due to retinal degeneration. The basic idea is to implant a microelectronic stimulation chip into a patient's eye, which replaces the lost vision by electrical stimulation of nerve cells.

There are two different approaches how to implement such retinal prostheses.

The subretinal approach, as for example described in the aforementioned WO 2005/000395 A1 and, for example, in EP 0 460 A2, uses a stimulation chip implanted in the subretinal space between the outer retina and the pigment epithelium of the retina, which converts ambient light incident on an array of photodiodes, which is integrated in the stimulation chip, into electrical stimulation signals for nerve cells. These stimulation signals drive an array of stimulation electrodes that stimulate the retinal neurons with spatially resolved electrical stimulation signals corresponding to the image information “seen” by the array of photodiodes.

This retinal implant stimulates the remaining intact neurons of the degenerated retina, i.e. horizontal cells, bipolar cells, amacrine cells and possibly also ganglion cells. The visual image falling onto the array of photodiodes or more complex elements is converted into an electrical stimulation pattern by the stimulation chip. This stimulation pattern then leads to the electrical stimulation of neurons, from which the stimulation is then directed to the ganglion cells of the inner retina and from there via the optic nerve into the visual cortex.

In other words, the subretinal approach uses the natural interconnection of the previously existing and now at least partially degenerated or lost photoreceptors with the ganglion cells to supply the visual cortex in the usual manner with nerve impulses that correspond to the seen image. The implant is thus a replacement for the lost photoreceptors and, like them, converts image information into electrical stimulation patterns.

On the other hand, the epiretinal approach uses a device consisting of an extra-ocular part and an intra-ocular part that communicate with each other in a suitable manner. The extra-ocular part comprises a kind of camera and a microelectronic circuit to encode the captured light, i.e. the image information, and transmit it as a stimulation pattern to the intra-ocular part. The intra-ocular part comprises an array of stimulation electrodes that contact neurons of the inner retina and thus directly electrically stimulate the ganglion cells located there.

The transmission of the electrical stimulation signals from the stimulation electrodes to the contacted cells, which is necessary for these implants, requires special attention. The coupling between a stimulation electrode and the contacted tissue is largely capacitive in nature, so that only transient signals can be used for electrical stimulation. This capacitive coupling is based on the fact that a capacitance (Helmholtz double layer) forms at the interface between electrode and electrolyte in the eye as a result of electrode polarization.

Against this background, the stimulation signals of known retinal implants are transmitted as square-wave monophasic or biphasic signal pulses with a certain repetition frequency, amplitude and pulse duration.

In the subretinal implant according to the aforementioned WO 2005/000395 the incident light is for example converted into voltage pulses with a pulse length of approx. 500 microseconds and a pulse interval of preferably 50 milliseconds, resulting in a repetition frequency of 20 Hz, which has proven to be sufficient for flicker-free vision. The pulse distance is also sufficient to completely restore or reset electrode polarization. It is mentioned that 20 Hz corresponds to the physiological flicker frequency at low ambient brightness.

Humayun et al., “Pattern Electrical Stimulation of the Human Retina”, Vision Research 39 (1999) 2569-2576 report on experiments with epiretinal stimulation using biphasic pulses having a cathodic phase, an intermediate phase and an anodic phase of 2 milliseconds each. At a stimulation frequency between 40 and 50 Hz, i.e. significantly above the physiological flicker frequency, a flicker-free perception could be observed in two patients.

Jensen and Rizzo, “Responses of ganglion cells to repetitive electrical stimulation of the retina”, Journal Neural Eng 4 (2007), S1-S6, report on subretinal stimulation experiments on an isolated rabbit retina with biphasic current pulses of different duration and repetition frequency. Already at a pulse interval of less than 400 milliseconds, they observed a decrease in stimulated activity for the following pulse.

WO 2007/128404 A1 addresses the question of how perception can be further improved by suitable selection of pulse duration and repetition frequency of the electrical stimulation signals.

The inventors report experiments in which the retina of a blind patient was stimulated subretinally with an electrode with biphasic, anodically starting pulses of up to 4 milliseconds duration. Using different repetition frequencies, i.e. excitation with a continuous sequence of “flashes” of a certain frequency, the following observation was made:

At higher frequencies about above 10 Hz the patient only felt flashes for a short time, after that the perception of the flashes subjectively disappeared. With an electrical stimulation having an average frequency below 10 Hz, however, the stimulus pulses were perceived as separate flashes for at least a few seconds. At frequencies of a few Hz and below, however, each flash was perceived as an individual flash, the sensation remains stable for minutes.

In view of these experimental findings with implanted subretinal implants, WO 2007/128404 A1 proposes to divide the multitude of stimulation electrodes into at least two groups of stimulation electrodes, which are controlled to emit stimulation signals in a time sequence one after the other.

The seen image is therefore not provided as a whole to the stimulation electrodes with a high repetition frequency; instead, the image is divided into at least two sub-images, so to speak, which are alternately “passed on” to the stimulation electrodes with a lower repetition frequency.

For example, if four partial images are respectively output at a repetition frequency of 5 Hz as stimulation signals of one quarter of the stimulation electrodes respectively, a new (partial) image in the form of stimulation signals, i.e. pulses, from the stimulation electrodes to the cells of the retina is nevertheless output at a partial image frequency of 20 Hz.

This may slightly reduce the spatialresolution, however the 20 Hz refresh rate required for physiologically flicker-free vision is still achieved.

Depending on the number and spatial “density” of the stimulation electrodes, it is also possible to use a larger number of partial images, if the desired spatial resolution can thus be achieved. With a higher number of partial images, the repetition frequency of the individual partial image can be further reduced, whereby still every 50 milliseconds, i.e. with a frame repetition frequency of 20 Hz, a new partial image in the form of a pattern of stimulation pulses is output.

Im and Fried, “Temporal properties of network-mediated responses to repetitive stimuli are dependent upon retinal ganglion cell type”, Journal Neural Eng 13 (2016), 1-12, report on epiretinal stimulation experiments on an isolated rabbit retina with five consecutive monophasic sinusoidal cathodic stimulation pulses of 4 milliseconds duration and resting intervals of 10 to 1000 milliseconds. Their results confirm the optimum repetition frequency of 5 to 7 Hz reported by users.

Weitz et al., “Improving the spatial resolution of epiretinal implants by increasing stimulus pulse duration”, Science Translational Medicine 7 (318), 318ra203, 16 Dec. 2015, report that with epiretinal stimulation a pulse duration of 25 milliseconds compared with shorter pulse durations enables the patient to recognize images with higher resolution. They mention that sinusoidal stimulation pulses of 20 Hz bipolar cells in blind ex-vivo retina stimulate more effectively than square-wave pulses of the same frequency.

Twyford and Fried, “The retinal response to sinusoidal electrical stimulation”, IEEE Transactions on Neural Systems and Rehabilitation Engineering, TNSRE-2014-00035.R2, discuss that in retinal implants and many other neuronal implants rectangular pulse shapes are used for the stimulation signals. They report on epiretinal experiments on seeing ex vivo retinas with sinusoidal stimulation pulses of 5, 10, 25 and 100 Hz, which were continuously applied for 5 seconds each, and led to measurable results. However, the authors conclude that the use of low-frequency sinusoidal stimulation signals in retinal prostheses may not make sense, since pharmacological experiments indicate that the stimulation takes place via photoreceptors, which are no longer present in a blind retina.

US 2012/0083861 A1 describes the use of low frequency sinusoidal stimulation signals (LFSS) to selectively stimulate ganglion cells. For the excitation frequencies, values below 100 Hz and below 25 Hz, for example 25 Hz and 5 Hz are provided. In the experiments performed, a stimulation electrode was positioned epiretinally. The authors discuss that by using LFSS lower requirements on the positioning accuracy of the stimulation electrode apply. They therefore see further advantages for the subretinal arrangement. The authors also speculate that certain subgroups of ganglion cells or bipolar cells could be selectively activated by appropriate selection of frequencies using LFSS.

In general, the stimulation electrodes of the electrical retinal implant are placed epi- or subretinally in close contact with the tissue to be stimulated in the eye to transmit the stimulation signals. The stimulation signals should be selected in such a way that the patient is able to see as flicker-free as possible with a correspondingly high temporal resolution, so that he can not only capture the environment (orientation vision) quasi-statically but also rapidly changing environmental impressions.

In order to stimulate the nerve cells in the vicinity of the electrodes, known implants apply short (approx. 0.1 to 10 milliseconds long) rectangular electrical voltage or current pulses with a certain repetition frequency (approx. 5 to 100 Hz) applied to the electrodes. This results in rest periods of approx. 10 to 200 milliseconds between the individual stimulation pulses. Sinusoidal stimulation impulses were also suggested in isolated cases.

However, in such a retinal stimulation a “fading” is observed, which means that the stimulated neurons are not stimulated by every pulse. The aforementioned WO 2007/128404 A1 tries to mitigate this problem by the described partial image overlay, in which each partial image, i.e. each assigned area of neurons is stimulated with a repetition frequency of 5 Hz.

This enables orientation vision, which is a great step forward for blind patients. However, the aim is to also enable the patient to acquire high-resolution images of rapidly changing scenes, e.g. while walking or watching television, which is not yet satisfactorily possible with the retinal implants currently in use.

Moreover, known implants apply voltage amplitudes greater than 1 Volt to reliably stimulate the neurons. This voltage value can lead to electrode damage, as it is outside the so-called “water window” in which no or very low chemical surface reactions take place. Voltages of 1.6 volts (with monophasic stimulation) can also lead to cell electroporation, i.e. damage to the stimulated cell membrane.

Another problem with the known retinal implants is the energy supply of the stimulation chip.

The energy for generating the electrical stimulation signals can also for subretinal implants not be obtained from the incident useful light itself, so that additional external energy is required. This external energy is either harvested from additional non-visible light radiated into the eye, externally for example inductively coupled via a coil, or conducted into the eye via a cable.

The implant described in WO 2005/000395 A1 is supplied with electrical energy wirelessly via irradiated IR light or inductively coupled HF energy, whereby this externally supplied external energy may contain information for controlling the implant.

However, as wireless retinal implants for human applications are not yet available in satisfactory quality, both epiretinal and also subretinal implants are currently used, to which the required external energy is supplied via cable.

For example, WO 2007/121901 A1 describes a subretinal retinal implant in which the external energy and control signals are transmitted via cable to the stimulation chip implanted in the eye. The cable is applied and fixed to the sclera of the eye to avoid forces on the implant.

Because implants, on the one hand, usually feature integrated circuits that are operated with DC voltage, and on the other hand, there is little space available on the implants themselves, most known implants are directly supplied with DC voltage. With an AC voltage supply, the rectifiers required on the implant may take up too much space, in particular because of the required smoothing capacitors, or may not be technically reasonably implemented in integrated circuits.

However, the cable-bound transmission of DC voltage leads to electrolytic decomposition processes in the tissue surrounding the cables in the long term, so that this method of supplying implants with external energy is also unsatisfactory.

WO 2008/037362 A2 therefore proposes to provide the implant with at least one essentially rectangular electric alternating voltage, which with respect to the tissue mass is on temporal average almost free of direct current. The potential level can be selected in such a way that the supply voltage is on average over time at least almost DC-free. Thereby, the disturbing electrolytic decomposition processes are at least largely avoided.

Despite the promising approaches described above for solving the major technological challenges associated with epi- and especially subretinal retinal implants, the currently available retinal implants may not yet meet all the requirements for comprehensive and satisfactory patient care.

It may still be investigated whether the epi- or subretinal approach is suitable for all patients suffering from visual impairment due to loss of natural photoreceptors, as is the case with retinitis pigmentosa or age-related macular degeneration.

Another requirement for retinal implants can be seen in that a robust neuronal activity is triggered with the lowest possible stimulation intensity (voltage or current amplitude). Robust, reliable stimulation refers to the ability of stimulated neuronal tissue to generate an electrical response each time the stimulus is presented.

An aim of all electrical implants is also to reduce the voltage required for the robust activation of the cells. This can be achieved, for example, by using materials with low impedance, wherein the long-term stability of these materials still needs to be investigated.

### SUMMARY

Against this background, it would among other objects be desirable to provide a retinal implant that takes these observations into consideration and avoids or reduces disadvantages of the solutions according to the prior art.

According to an aspect of the present disclosure, there is provided an active retinal implant as mentioned above that has at least one signal generator which generates at least one continuous sinusoidal stimulation signal comprising at least one adjustable signal parameter, and wherein the at least one signal generator is electrically coupled to at least one stimulation electrode to which it supplies the stimulation signal, wherein the signal parameter may be at least one selected from a group comprising frequency, amplitude, phase, offset and/or waveform.

According to an aspect of the present disclosure, the stimulation is effected with an array of stimulation electrodes which are operated with sinusoidal signals. These may be generated, for example, by at least one continuously operated sinusoidal signal generator. Amplitude, frequency, offset or phase relationship of the sinusoidal signals applied to the individual stimulation electrodes can be set individually and variable over time for each stimulation electrode or for groups of stimulation electrodes. These signal parameters may be adjusted based on the intensity of light incident on the retina and measured by photodiodes. An increased incidence of light may be converted into an increased stimulation amplitude or increased stimulation frequency. A suppression of the activity, as occurs when the visual field is darkened, may be achieved by a reduced stimulation amplitude or a phase-shifted stimulation. A shift of the voltage zero line, i.e. an offset, can also be used to take account of an increased or reduced incidence of light.

The stimulation parameter of the output stimulation signal can be the result of a mathematical calculation, whereby the calculation is either analog or digital.

A “sinusoidal stimulation signal” in the context of the present disclosure may refer to both a “pure” sinusoidal signal following the trigonometric formulae and a continuous signal derived, for example, mathematically from a pure sinusoidal signal which may be asymmetrical with respect to aspect ratio and/or time component of the positive and negative half-wave and/or ratio between the slopes of the positive and negative edges.

The stimulation signal output to neighboring stimulation electrodes can have an adjustable phase relationship, whereby the capacity of the stimulation electrodes can contribute to the adjustment of the phase of the stimulation signal.

After implantation and start-up of a retinal implant, the stimulation frequency of the sinusoidal stimulation signals may be individually adjusted so that the patient reports stable visual impressions. The used stimulation electrodes may be metal-based or based on a capacitive material. When capacitive electrodes are used, sinusoidal stimulation results in a phase shift.

According to an aspect of the present disclosure, a reduction of the voltage required for a robust activation of the cells may not achieved by the use of previously untested materials with low impedance, but by a new stimulation protocol that allows the use of already established materials.

The new implant comprises at least one signal generator, which in the simplest case comprises a sinusoidal signal generator with certain signal parameters that can be adjusted via external control parameters. The implant can also have several sinusoidal signal generators or a complex signal generator comprising current sources, digital-to-analog converters, microcontrollers, etc. to generate stimulation signals of any waveform, frequency, amplitude and phase relationship.

The systems described in the two publications discussed below may be used to carry out the teachings disclosed herein if they are adapted accordingly.

U.S. Pat. No. 6,591,138 B1 describes a control device that can be implanted in the brain of a patient, which measures the electrical activity of the brain and, for certain measurement signals, emits sinusoidal stimulation signals with a fundamental frequency below 10 Hz into the brain via implanted electrodes. The stimulation signals may vary in terms of frequency, phase, waveform, duration and amplitude from electrode to electrode. Thereby, undesirable neurological conditions shall be prevented or terminated.

Ghovanloo and Najafi, “A wireless implantable multichannel microstimulating system-on-a-chip with modular architecture”, IEEE Transactions on Neural Systems and Rehabilitation Engineering, vol. 15, No. 3, September 2007, describe a microsystem conceived e.g. as an epiretinal implant that can deliver mono- or biphasic stimulation pulses to up to 64, in a further development to up to 2048 stimulation points. The implant is constructed monolithically as an ASIC and allows the generation of any waveform for the stimulation pulses as well as extensive adjustment of the parameters of the individual stimulation signals.

Stimulation with an array of stimulation electrodes that are individually provided with a sinusoidal stimulation signal may enable a reduction of the stimulation voltage required for stable stimulation. This protects both the stimulation electrodes and the stimulated tissue.

Furthermore, an increase of the stimulation frequency beyond the previously optimal range of 5-7 Hz may become possible. The “fading” described by patients, i.e. the decay of the stimulated electrical activity, may be mitigated or completely eliminated by increasing the stimulation frequency to at least 10 Hz.

The application of stimulation signals with different frequencies and phase relationships to each other can enable a site- or location-specific stimulation.

According to a refinement of the present disclosure, the stimulation may be carried out with low current or voltage amplitudes, the maximum voltage amplitude of each half-wave may now be less than 1 Volt, in particular in a range of 50 mV to 300 mV.

At a voltage amplitude of, for example, 100 mV per half-wave the stimulation signal has a total voltage swing of 200 mV, which can be symmetrical to a zero line, or can also have an offset if the zero line has been shifted.

Furthermore, continuous stimulation with a frequency greater than 10 Hz is possible, in particular in the range of 10-100 Hz.

At this stimulation frequency, the desired stimulation effect may occur permanently each time a stimulation sequence is repeated. Consequently, subretinal stimulation may not result in fading.

Further, the present disclosure also provides the possibility to apply complex spatio-temporal stimulation patterns in order to maximize the physiological effect individually.

In this way, an advantageous retinal implant may be provided.

It is advantageous if the signal parameter is individually adjustable for each stimulation electrode, whereby the signal generator may in particular comprises a dedicated sinusoidal signal generator for each stimulation electrode.

In a further refinement, an image receiver may be provided which converts incident ambient light into electrical signals which are fed to the at least one signal generator in order to influence or adjust the at least one signal parameter.

The electrical signals may thus comprise the required image information spatially resolved, based on which the signal generator may then be controlled in such a way that it outputs a pattern of electrical stimulation signals with signal parameters set accordingly for the individual stimulation electrodes, so that the stimulation signals may stimulate the neurons in such a way that the patient can see the image “received” by the image receiver.

In a refinement, the image receiver may comprise an array of image cells, wherein each image cell is associated with a stimulation electrode, and wherein the electrical signal generated by an image cell is used to adjust the signal parameters of the stimulation signal, which is supplied to the associated stimulation electrode.

Each image cell may thus influences the signal parameters of the stimulation signal that is fed to the associated stimulation electrode.

The image receiver may be implemented as an external image sensor or receiver, which is arranged outside the eye.

The externally recorded and processed image information may transmitted to the implant in the form of electrical signals via a cable or wirelessly, as in the well-known epiretinal implants. There, the signals are optionally further processed and then used as an “internal image” to control the signal generator(s) in such a way that the signal parameters of the stimulation signals are adapted in such a way that the neurons are then stimulated via the stimulation electrodes in such a way that the seen image can be recognized.

Design or constructive details of the external image receivers, the processing electronics and the “data transmission” into the eye can be taken over from the known epiretinal implants—if necessary with appropriate adaptation.

Alternatively, the image sensor or receiver can also be implemented as an implantable image receiver that is also implanted in the eye.

This alternative may offer a significant advantage.

With an image receiver mounted on the outside of the eye, it is not possible to use the eye movement, which fulfills an important function in finding objects. The patient would always see the same image despite different eye positions as long as the head is held still. This is confusing for him and would, according to insights of the inventors, reduce the benefit of the implant. It has already been suggested to use a so-called eye-tracking control for external image receivers, wherein the eye movement may be detected and used. However, this approach proves to be very complex, whereby no experience is yet available as to whether this will be possible with sufficient accuracy.

However, if the image receiver is also implanted into the eye, the patient can use the natural eye movement and head movement in the usual way to see images and scan or search for objects.

Design details of the implanted arrays of photodiodes, the control and processing electronics and the energy transfer into the eye—if necessary with appropriate adaptation—can be adopted from the subretinal implants mentioned above, which is why the disclosure of the aforementioned IP rights is hereby made subject of the present application by reference.

The new implant may thus be used sub- or epi-retinally.

In a refinement, the image receiver and the signal generator(s) may be integrated in a chip.

An advantage of this embodiment may be that the new chip may be easier to implant than an implant consisting of two chips or components.

In general, it may be advantageous for the implant to include a switch-off device that switches at least the signal generator or the signal generators on and off based on external control signals.

An advantage of this embodiment may be that the effect of closing the eyelids can also be achieved with continuous stimulation signals.

Further advantages are apparent from the description and the appended drawings.

It will be appreciated that the features specified above and those still to be elucidated hereinafter can be used not only in the particular combination indicated, but also in other combinations or on their own, without departing from the scope of the present invention.

### BRIEF DESCRIPTION OF THE DRAWINGS

Exemplary embodiments of the disclosure are shown in the drawings and is elucidated in more detail in the description hereinafter. The drawings showing

FIG. 1 a schematic representation of a first exemplary embodiment of he new retinal implant, wherein the representation not drawn to scale;

FIG. 2 a schematic representation of a second exemplary embodiment of the new retinal implant, wherein the representation not drawn to scale;

FIG. 3 a schematic representation of a human eye into which the retinal implant according to FIG. 2 is inserted, wherein the representation is also not drawn to scale;

FIG. 4 a schematic representation of a third exemplary embodiment of the new retinal implant, wherein the representation is not drawn to scale;

FIG. 5 a schematic representation of a signal generator, as used in the implants of FIG. 1 to 4, for generating sinusoidal stimulation signals from the received image signals with corresponding signal parameters;

FIG. 6 a sinusoidal signal waveform with asymmetric aspect ratio that can be used for the stimulation signals;

FIG. 7 a further sinusoidal signal waveform with asymmetrical gradients that can be used for the stimulation signals;

FIG. 8 a summary of the results of a 5-minute subretinal continuous wave stimulation of a blind ex vivo retina with a sinusoidal excitation signal of 25 Hz;

FIG. 9 a summary of the results of a 5-minute subretinal continuous wave stimulation of a blind ex vivo retina with a sinusoidal excitation signal of 40 Hz;

FIG. 10 a summary of the results of a 5-minute subretinal continuous wave stimulation of a blind ex vivo retina with an asymmetric excitation signal of 120 ms period; and

FIG. 11 a summary of the results of a 5-minute epiretinal continuous wave stimulation of a blind ex vivo retina, alternatingly using a sinusoidal excitation signal of 10 Hz and a sinusoidal excitation signal of 25 Hz.

### DESCRIPTION OF PREFERRED EMBODIMENTS

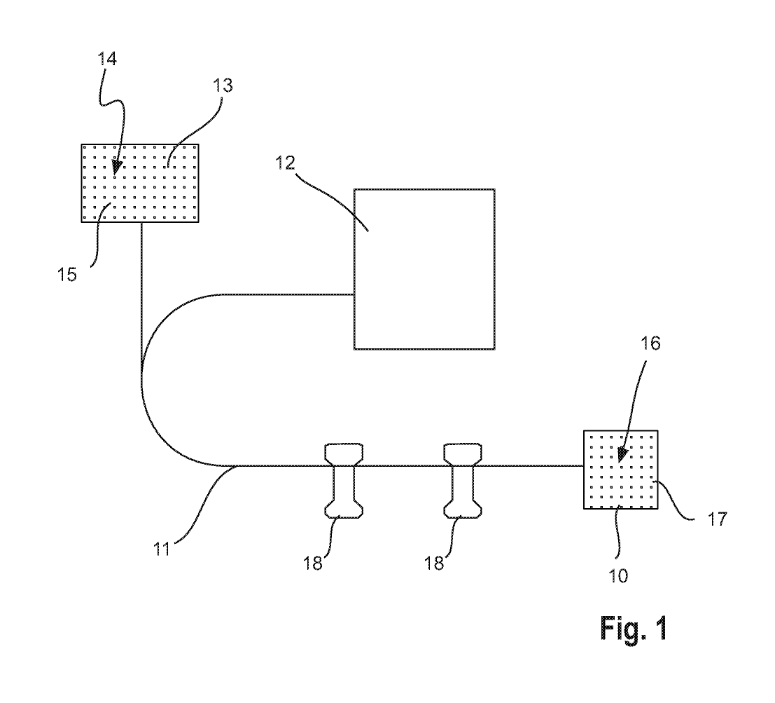

FIG. 1 schematically shows a first embodiment of an active retinal implant 10, whereby the dimensions are not drawn to scale.

The retinal implant 10 is connected via a cable 11 to a supply unit 12 and to an implantable image receiver 13, on which an array 14 of image cells 15 is arranged, which are, for example, implemented as photodiodes. An array 16 of stimulation electrodes 17 for emitting electrical stimulation signals is arranged on the retinal implant 10.

The supply unit 12 supplies the retinal implant 10 with electrical energy via cable 11 and, if necessary, with control signals via which various functions of the retinal implant can be influenced or adjusted.

The image receiver 13 converts incident ambient light via its image cells 14 into spatially resolved electrical signals, which are transmitted to the retinal implant 10 and—if necessary after further processing—are provided via the stimulation electrodes 17 as electrical stimulation signals to retinal cells for stimulating them.

The retinal implant 10 may be used epi- or sub-retinally.

The cable 11 is provided with attachment straps or leaches 18, with which the cable 11 can be attached to the sclera of the eye of the person to whom the retinal implant 10 is implanted. This prevents forces from being exerted on the retinal implant 10 that could lead to mechanical loading and/or displacement of the retinal implant 10.

Regarding the retinal implant 10 of FIG. 1, the image receiver 13 is arranged outside the eye, e.g. in spectacles worn by the patient. The retinal Implant 10 is then implanted, for example, epiretinally whereby the transmission of energy, control signals and image information can also take place wirelessly.

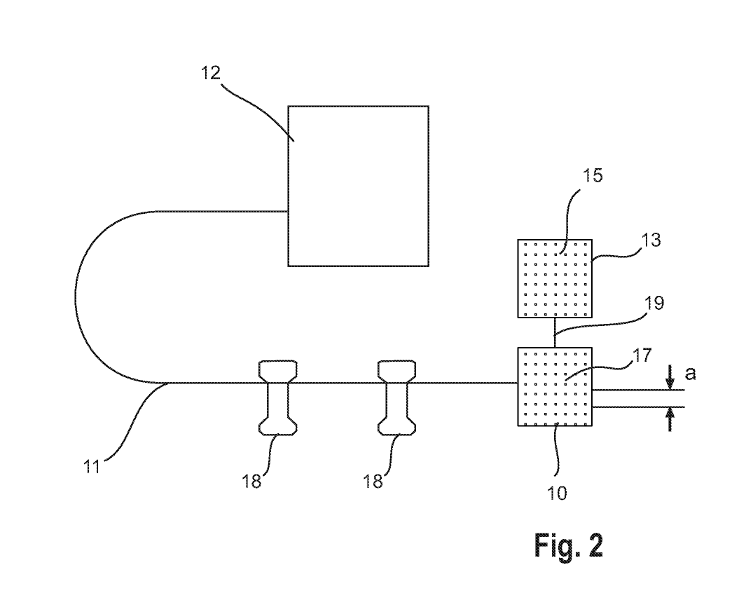

However, in preferred exemplary embodiments, the image receiver 13 is adapted to be implantable such that, like the retinal implant 10 itself, it is implanted into the eye. This embodiment is shown in FIG. 2, wherein the image receiver 13 is arranged next to the retinal implant 10, to which it is connected, in the shown exemplary embodiment, via a cable 19.

Since the image receiver is also implanted into the eye with this subretinal implant, the patient can use natural eye movement and head movement in the usual way to see images and scan for objects.

Design or construction details of the implanted arrays of photodiodes, the control and processing electronics as well as the energy transfer into the eye can be taken over—if necessary with appropriate adaptation—from the subretinal implants mentioned above.

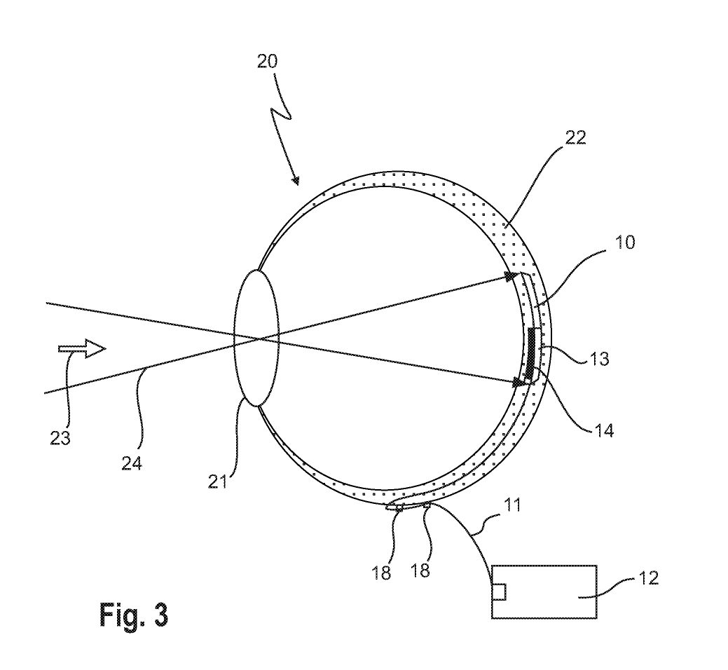

The retinal implant 10 and the image receiver 13 from FIG. 2 are adapted to be implanted in a human eye 20, which is shown very schematically in FIG. 3. For simplicity, only the lens 21 and the retina 22 are shown, into which the implant 10 and the image receiver 13 have been implanted.

Retinal implant 10 and image receptor 13 are preferably placed in the so-called subretinal space that forms between the pigment epithelium and the photoreceptor layer. If the photoreceptor layer is degenerated or lost, the subretinal space forms between the pigment epithelium and the layer of bipolar and horizontal cells. The retinal implant 10 is placed so that the stimulation electrodes 17 shown in FIG. 2 can transmit the electrical stimulation signals to cells in the retina 22.

Visible light indicated by arrow 23, the ray path of which is shown at 24, is transmitted via the lens 21 to the image receiver 13, where the visible light (see arrow 23) is converted into electrical signals, which are transmitted to the retinal implant 10 and converted there into electrical stimulation signals.

Retinal implant 10 and implantable image receiver 13 can be arranged next to each other as shown in FIG. 2, wherein they can be implemented as separate units, for example, using different technologies. Both implants 10, 13 can also be arranged next to or above each other on a common foil or can be integrated into a microchip.

The array 14 of image cells 15 is shown in FIG. 3 as a black area.

The stimulation electrodes 17 are provided in a defined geometric arrangement and have a separation of 50 μm from each other, which is indicated by ‘a’ in FIG. 2.

This arrangement can be matrix-shaped with rows and columns—as arranged in FIGS. 1 and 2—or beam-shaped in order to be able to generate different patterns that ensure optimum detection or recognition.

In FIG. 3 it can further be seen that the cable 11 is led out of the eye at the side and fastened there outside on the sclera with the fixing straps 18, before the cable continues to the external supply unit 12.

The supply unit 12 is then for example attached to the patient's skull outside the eye in a manner not shown in further detail. Electrical energy is sent to implant 10 and image receiver 13 via supply unit 12, wherein at the same time, control signals can be transmitted which influence the functioning of the implant as described, for example, in the aforementioned WO 2005/000395 A1, the content of which is hereby made subject of the present application.

The energy supply can be effected via essentially rectangular AC electrical voltages which on temporal average are almost free from direct current with respect to the tissue mass, as described in the aforementioned WO 2008/037362, the contents of which are also made subject of the present application.

It should also be mentioned that the dimensions of the retinal implant 10, the image receptor 13, the fixing straps 18 and the external supply unit 12 in FIGS. 1, 2 and 3 are shown neither to scale nor in the correct size relation to each other.

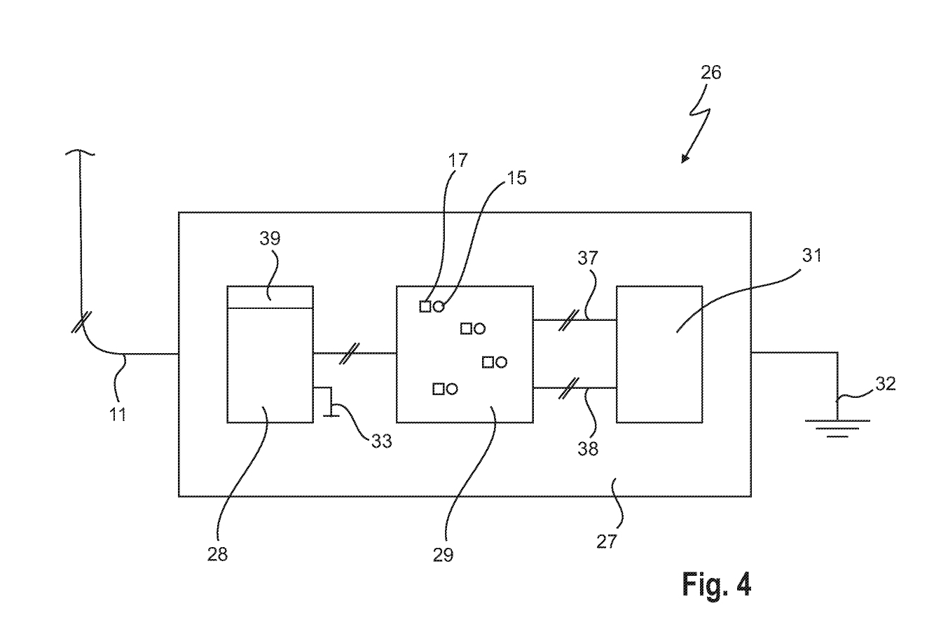

Alternatively, the image receiver 13 and the retinal implant 10 may also be integrated in a chip 26, as schematically shown in FIG. 4. The chip 26 is easier to implant than an implant consisting of two chips or components. Furthermore, the location where the image information is acquired (by the image receiver 13) and the location where the electrical stimulation signals are provided are very close together, so that the patient does not notice any or almost no prism errors.

The chip 26 comprises a carrier 27 on which an input stage 28 can be seen, to which external energy and optionally control signals are provided via cable 11. The input stage 28 is coupled to a unit 29, which in this case has a plurality of image cells 15, which converts incident visible light into electrical signals, which are then provided as electrical excitation patterns to nerve cells of the retina via the stimulation electrodes 17 indicated next to the respective image cells 15.

The electrical signals generated by the image cells 15 are processed by a signal generator 31, which generates sinusoidal stimulation signals with individual signal parameters for the various stimulation electrodes 17, which are then provided to the stimulation electrodes 17.

In this context it should be pointed out that FIG. 4 is only a schematic representation of the chip 26 which reflects the logic structure. The actual geometric arrangement of the individual components can lead, for example, to each image cell 15 having a signal generator 31 in the immediate vicinity.

The chip 26 is connected to the tissue into which the implant is inserted via an external mass indicated by 32. Furthermore, an internal electrical ground 33 is indicated, which in the exemplary embodiment is not connected to the external ground 32.

As an alternative to the wired power supply described so far, the chip 26 can also be supplied inductively via an external transmitter coil with RF energy which is received by a receiving coil in/at the eye and then rectified to supply the chip 26 with the required DC voltage, as for example described in WO 2009/090047A1.

The chip can also be supply with energy via infrared radiation, as described in detail in WO 2004/067088 A1, the content of which is also made subject of the present application.

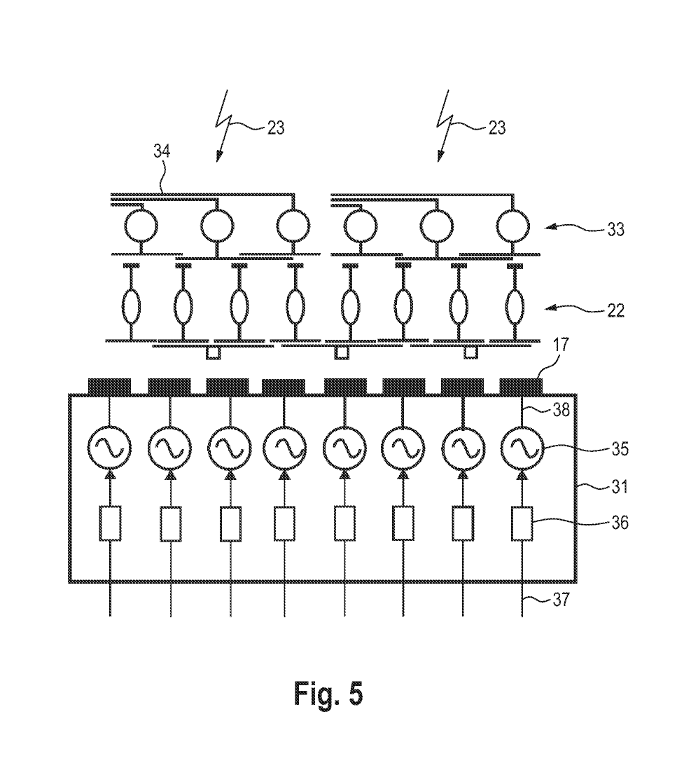

FIG. 5 shows an example of a signal generator 31, opposite of which there are very schematically indicated a retina 22 followed by neuronal tissue 33, which is connected via nerve paths 34 to the visual cortex not shown. In an intact retina 22, incident light (see arrow 23) is converted into electrical signals by the retina 22 and emitted to cells of neuronal tissue 33, which then transmits the electrical signals via the nerve pathways 34 to the visual cortex.

The function of the retina 22, which is not or no longer fully functional for the respective patients, is performed by the retinal implant 10 according to an aspect of the present disclosure.

The signal generator 31 comprises a sinusoidal signal generator 35 for each stimulation electrode 17, that generates a sinusoidal stimulation signal with adjustable signal parameters.

Each sinusoidal signal generator 35 is assigned a setting device 36, to which the electrical signals output by the image cells 15 are provided via lines 37, wherein the electrical signals represent the seen image.

The electrical signals of the image cells 15 influence frequency, amplitude, relative (related to stimulation signals for adjacent stimulation electrodes) phase and/or relative (again related to stimulation signals for adjacent stimulation electrodes) offset of the respectively generated stimulation signal, which is then transmitted via lines 38 to the stimulation electrodes 17.

The signal generator 31 can, depending on the individual signal parameters determined by the electrical signals on the lines 37, also generate stimulation signals with the aid of current sources, analog-to-digital converters, microprocessors, etc.

The lines 37, 38 are also shown in FIG. 4, where double dashes indicate that several lines 37 and 38 are provided respectively.

For example, the patient may actively switch the retinal implant 10 on and off via control signals on cable 11 to achieve the same effect (dark state) as when closing the eyelids during rest phases or during the night. By the continuous output of sinusoidal stimulation signals to the stimulation electrodes 17 the patient may otherwise be provided with a permanent “basic image” that does not disappear even when the eyelids are closed.

The switch-off device 39 provided for this purpose is indicated in FIG. 4 by a rectangle in the input stage 28. The switch-off function can be implemented as a hardware module or as a software module.

During operation, the frequencies of the stimulation signals, that are provided to the individual stimulation electrodes 17 or groups of stimulation electrodes 17, can be individually adjusted so that the patient perceives a uniform visual impression over the entire stimulated image.

According to the findings of the inventors, this individual frequency adjustment may allow that the different physiologic conditions of the individual patients to be taken into account, so that all stimulation electrodes 17 provide their stimulation signals to the respective associated cells of the neuronal tissue 33.

Without being limited thereto, at the present stage an explanation for this effect is that by changing the sinus frequency of the stimulation signal, the neuronal tissue cells addressed by the stimulation signal can be selected.

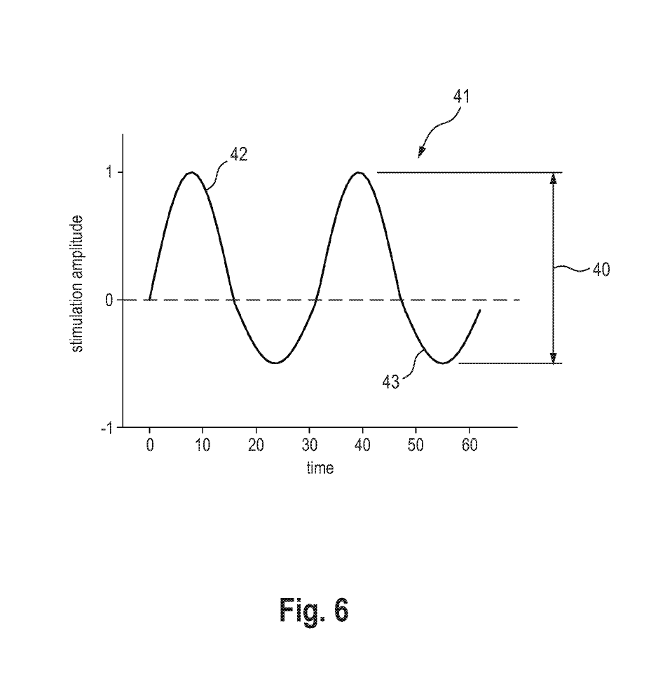

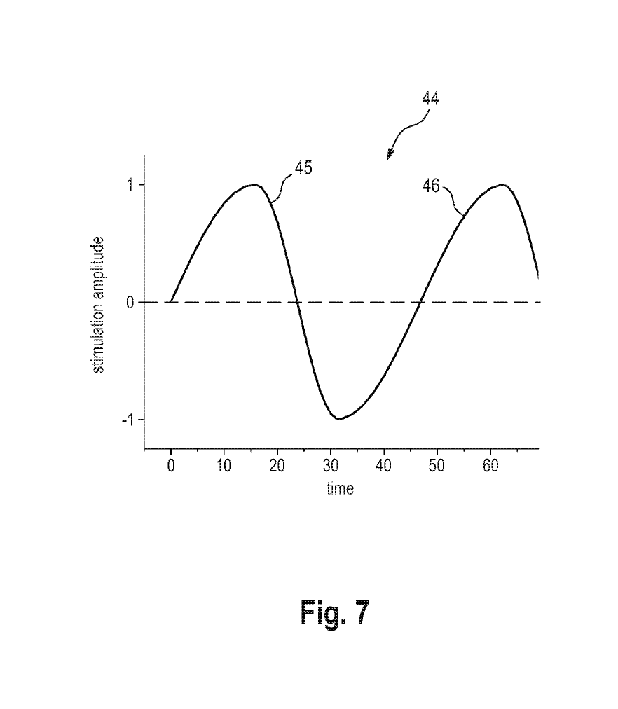

Instead of purely trigonometric sinusoidal signals, stimulation signals deviating from the pure sinusoidal shape may also be used, as exemplarily shown in FIGS. 6 and 7.

FIG. 6 shows a sinusoidal signal 41 with an amplitude swing 40 having an asymmetrical aspect ratio, in which the anodic component 42 has a different, here larger amplitude than the cathodic component 43.

This takes into account the finding that in epi- and subretinal stimulation anodic signal components may have a different stimulation threshold than cathodic signal components.

Thereby a more effective stimulation can be provided without having to increase the amplitude swing 40.

FIG. 7 shows a sinusoidal signal 44 with asymmetrical gradients or slopes, in which the falling edge 45 has a different gradient, here greater, than the rising edge 46.

This takes into account the finding that in epi- and subretinal stimulation the stimulated activity—possibly depending on the stimulation frequency—is primarily associated with one of the two flanks.

Sinusoidal signals with asymmetry regarding the aspect ratio and/or slope can be generated by simple mathematical operations from purely trigonometric sinusoidal signals.

Measurements by the inventors of the present application have revealed that blind retinas can be stimulated with sinusoidal electrical voltages via subretinally and via epiretinally positioned electrodes. The experiments included the recording of ganglion cell activity and the recording of the total current flowing during stimulation for later determination of the charge transferred per stimulation phase. For comparison with stimulation parameters used in clinical applications, retinas were stimulated with biphasic stimulation pulses of same duration as well as with short, pulse-shaped anodic stimulation pulses of the same frequency.

The retina of a blind mouse line (rd1, 8 weeks old, reference: Charles River) served as a reference, wherein the retina was apply to a microelectrode array (MEA) in subretinal or in epiretinal configuration. During the measurements the retina was perfused with gasified (carbogen) Ame's medium (sigma). The temperature in the bath was kept at 37° C.

Two different MEA types with iridium oxide electrodes were used: (i) Standard MEAs with 200 μm electrode spacing and 30 μm electrode diameter and (ii) HD-MEAs (electrode spacing 30 μm, electrode diameter 10 μm). Simultaneous stimulation with 8 electrodes stimulated an electrode field of either 430 μm×430 μm or 70 μm×70 μm. The electrode material was iridium oxide. All stimulation voltages used were generated by an STG 2004 (Multi Channel Systems MCS GmbH). For sinusoidal excitation, a voltage divider was connected between STG 2004 and the MEA adapter in order to achieve the highest possible resolution. An external Ag/AgCl was positioned in the bath as a counter electrode.

The cell responses triggered by electro-stimulation were measured for subretinal stimulation using a flexible microelectrode array (Flex MEA). The stimulation current was determined by a resistance (10 Ohm) between Ag/AgCl electrode and system mass.

Sinusoidal stimulation signals with frequencies of 10 and 25 Hz were tested at signal amplitudes of 15, 50, 100, 150 and 200 mV. Stimulation of the retina with amplitudes of 200 mV was measurable for both frequencies.

Then it was estimated how stable the stimulated activity is over the stimulation period of 5 minutes, i.e. whether the activity triggered by the first pulses is comparable to the activity triggered by the last pulses. When comparing the first 18 and the last 18 seconds of the 5-minute stimulation with 200 mV amplitude and 25 Hz, there is no difference in the stimulated activity, i.e. no fading occurs here, as has been reported for example for pulse stimulation.

From the obtained results it could be concluded that sinusoidal continuous stimulation generates stable responses in which in 70% (and often 100%) of the stimulation cycles at least a retinal action potential is generated over 5 minutes.

Compared to excitations with rectangular stimulation pulses of the same frequency, the sinusoidal stimulation at 200 mV excitation amplitude resulted in significantly higher activity in the stimulated ganglion cells.

It could be shown that with subretinally applied sinusoidal voltages of amplitude 200 mV the blind retina can be reliably stimulated. On average, at a stimulation frequency of 10 Hz (25 Hz), 80% (70%) of all stimuli have at least 1 action potential/spike in the measured cell.

The stability of the induced response is higher than in the case of biphasic rectangular stimuli or after anodic, short pulses. With sinusoidal voltages more action potentials per stimulation can be triggered than with control stimuli.

Further experiments showed that even after 60 minutes of stimulation in both the epiretinal and subretinal approaches, the measured stimulation response did not decrease.

It was also shown that changes in stimulation frequency and stimulation amplitude are directly reflected in the stimulation response.

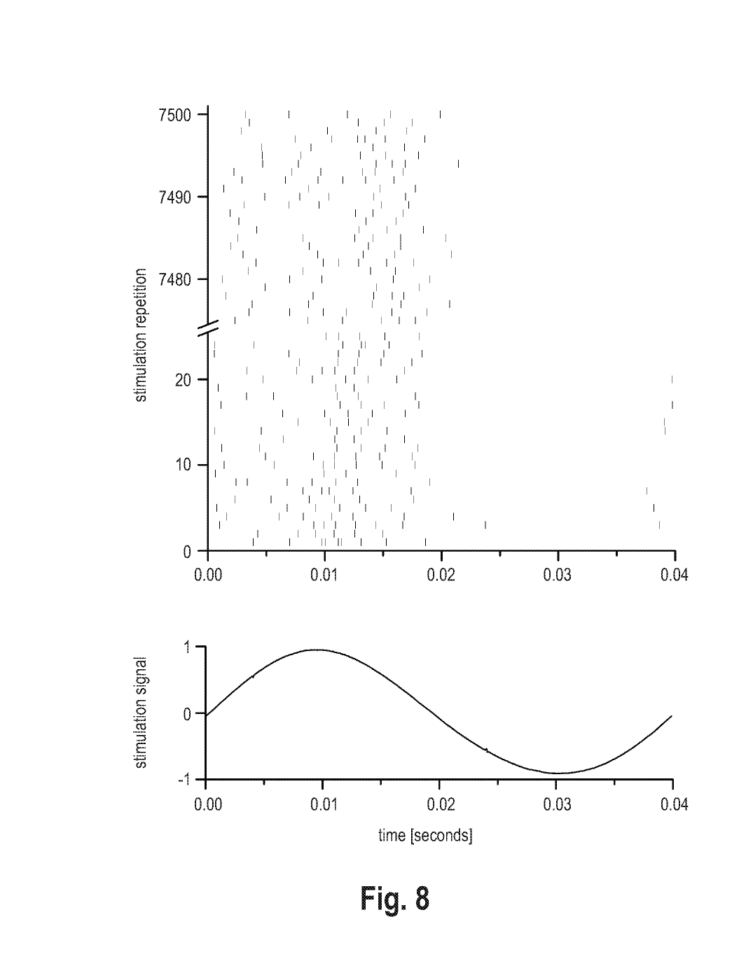

FIG. 8 shows the results of a 5-minute subretinal continuous wave stimulation of a blind ex vivo retina. Each line in the top graph corresponds to a measured action potential. The measurement results for the first and the last twenty sine curves are shown.

The bottom figure shows the transient or temporal variation of the excitation signal, which was a pure sine wave here with a frequency of 25 Hz and a total swing of 400 mV.

FIG. 8 firstly shows that the stimulation is robust, even after 5 minutes of continuous stimulation the action potentials are still reproducibly excited, no fading is recognizable.

Furthermore, it can be seen in FIG. 8 that the action potentials are mainly triggered in the anodic stimulation part. This result can, for example, be taken into account by asymmetrically designing the waveform of the stimulation signals, for example with a shorter cathodic component and/or steeper negative edge.

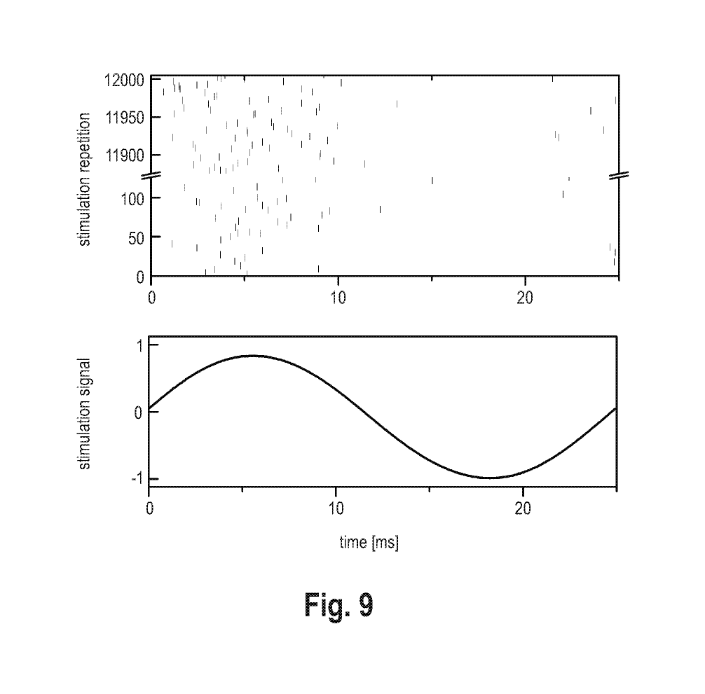

FIG. 9 shows in a diagram as in FIG. 8 the results of a 5-minute subretinal continuous wave stimulation of a blind ex vivo retina, but for an excitation signal that was a pure sine wave with a frequency of 40 Hz and a total swing of 400 mV.

FIG. 9 shows that the stimulation is robust even when stimulated with a 40 Hz sine wave. After 5 minutes of continuous stimulation, the action potentials are still reproducibly stimulated, whereby a fading is not recognizable here either.

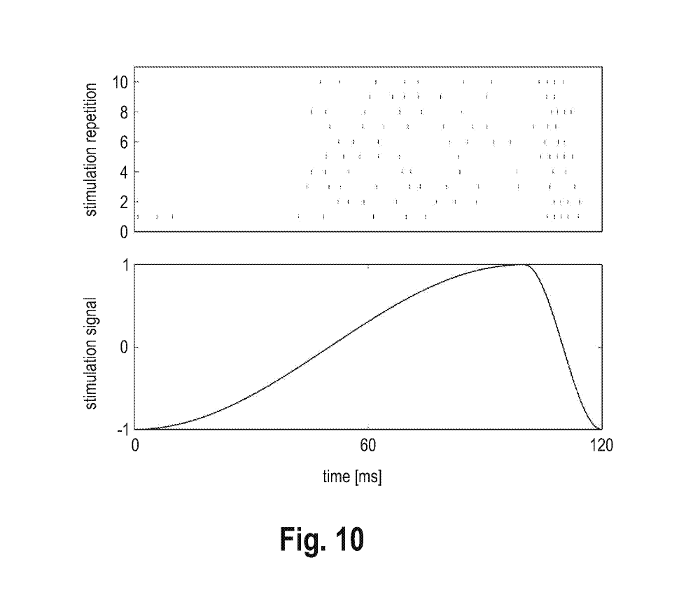

FIG. 10 bottom graph shows an asymmetrical excitation signal of 120 ms period duration and with steeply rising sinusoidal edge and flat falling edge; compare also with FIG. 7. The top graph in FIG. 10 shows the cell response, which is stimulated by the beginning of the falling edge of the excitation signal.

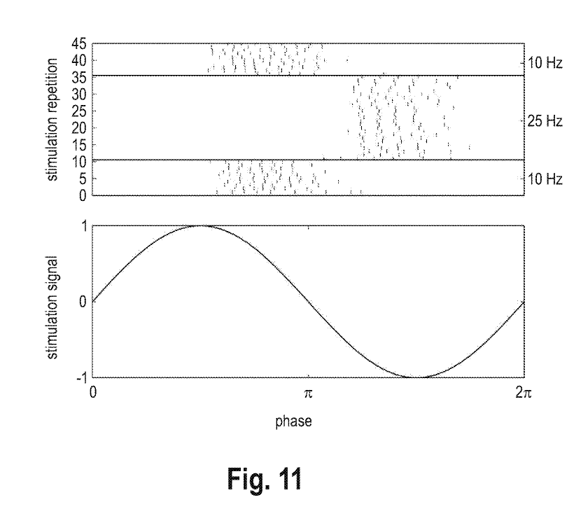

FIG. 11 shows, in a diagram as in FIG. 8, that the retinal network follows a change in frequency from 10 Hz to 25 Hz and back to 10 Hz. By increasing and subsequently reducing the excitation frequency, the cell responses are reproducibly shifted along the time axis. The number of triggered cell responses per second also changes, which represents a possibility for the conversion of the incident light signal into cell responses.

The results shown in FIG. 11 show that the cell response can be modulated within a few milliseconds by changing the stimulation frequency.

The experimental set-up and the evaluation of the measured values for the results shown in FIGS. 8 and 9 corresponded to the setting discussed above preceding the discussion of FIG. 8. The tests for FIGS. 8 and 9 were performed with subretinal stimulation, those for FIGS. 10 and 11 with epiretinal stimulation.

The experimental setup and the evaluation of the measured values for the results shown in FIG. 10 11 were obtained with a commercial CMOS-based microelectrode array “CMOS MEA 5000” (Multi Channel System MCS GmbH). The stimulation current was 500 nA. The electrode material was titanium nitride isolated with a 25 nm titanium oxide layer.

The cell responses triggered by electro-stimulation with CMOS MEA were measured for epiretinal stimulation using a sensor electrode of the CMOS MEA 5000 system. The stimulation current was determined by a resistance (10 Ohm) between Ag/AgCl electrode and system mass.

What is claimed is:

1. An active retinal implant for implantation in an eye, comprising an array of stimulation electrodes which deliver stimulation signals to cells of the retina,
wherein at least one signal generator is provided which generates at least one continuous sinusoidal stimulation signal comprising at least one adjustable signal parameter, and in that the at least one signal generator is electrically coupled to at least one stimulation electrode.

2. The retinal implant according to claim 1, wherein the signal parameter is selected from frequency, amplitude, phase, offset and/or waveform.

3. The retinal implant according to claim 1, wherein the signal parameter is individually adjustable for each stimulation electrode.

4. The retinal implant according to claim 1, wherein the signal generator comprises a dedicated sinusoidal signal generator for each stimulation electrode.

5. The retinal implant according to claim 1, wherein the at least one sinusoidal signal has a frequency greater than 10 Hz.

6. The retinal implant according to claim 5, wherein the at least one sinusoidal signal has a frequency in the range of 10 Hz to 100 Hz.

7. The retinal implant according to claim 1, wherein the at least one sinusoidal signal in each half-wave has an amplitude less than 1 Volt.

8. The retinal implant according to claim 7, wherein the at least one sinusoidal signal in each half-wave has an amplitude in the range of 50 mV to 300 mV.

9. The retinal implant according to claim 1, wherein an image receiver is provided which converts incident ambient light into electrical signals which are fed to the at least one signal generator to influence the at least one signal parameter.

10. The retinal implant according to claim 9, wherein the image receiver comprises an array of image cells, wherein each image cell is associated with a stimulation electrode, and wherein the electrical signal generated by an image cell is used to adjust the signal parameters of the stimulation signal which is supplied to the associated stimulation electrode.

11. The retinal implant according to claim 9, wherein the image receiver is an external image receiver which is arranged outside the eye.

12. The retinal implant according to claim 9, wherein the image receiver is an implantable image receiver.

13. The retinal implant according to claim 9, wherein the image receiver and the at least one signal generator is integrated in a chip.

14. The retinal implant according to claim 1, wherein it comprises a switch-off device which switches at least the signal generator on and off as a function of external control signals.
